(19) United States
(12) Patent Application Publication
(10) Pub. No.: US 2021/0060164 A1
(43) Pub. Date: Mar. 4, 2021

(54) NONINVASIVE DEEP BRAIN STIMULATION (DBS) USING X-RAY-EXCITED OPTICAL LUMINESCENT (XEOL) NANOMATERIALS (NANOSCINTILLATORS)

(71) Applicant: UCHICAGO ARGONNE, LLC, Chicago, IL (US)

(72) Inventors: Elena A. Rozhkova, Lemont, IL (US); Zhaowei Chen, Downers Grove, IL (US); Vassiliy Tsytsarev, Rockville, MD (US)

(21) Appl. No.: 16/552,576

(22) Filed: Aug. 27, 2019

(51) Int. Cl. A61K 41/00; A61N 5/10; A61N 5/06

(52) U.S. Cl. CPC A61K 41/0038; A61N 5/10; B82Y 5/00; A61N 2005/1085; A61N 5/0622

(57) ABSTRACT

A method for performing deep tissue stimulation using radiation-enabled optogenetics including introducing a plurality of nanoscintillators into a region with light sensitive cells, and targeting the nanoscintillators with a primary radiation to cause the nanoscintillators to emit optical energy. The light sensitive cells having an optically active wavelength range, and the emitted optical energy having a wavelength in the optically active wavelength range. In particular, introducing X-ray excited optical luminescent nanomaterials to a tissue region and controlling electrical activity in cells containing opsins.

### STATEMENT REGARDING FEDERALLY SPONSORED RESEARCH OR DEVELOPMENT

This invention was made with government support under Contract No. DE-AC02-06CH11357 awarded by the United States Department of Energy to UChicago Argonne, LLC, operator of Argonne National Laboratory. The government has certain rights in the invention.

### FIELD OF THE DISCLOSURE

The present disclosure relates to deep brain stimulation therapies, and more specifically to non-invasive deep brain stimulation using X-ray induced optical luminescence.

### BACKGROUND

Deep brain stimulation (DBS) is used to treat a number of neurological and psychiatric conditions, including, but not limited to, Parkinson's disease, Tourette syndrome, epilepsy, essential tremor, addictions, chronic pain, dementia, and obsessive-compulsive disorders. Electrical stimulation of the brain tissue is a conventional medical procedure used to treat several disabling neurological symptoms. Typically, this involves implanting electrodes within certain regions of a patient's brain to administer an electric current to the specific region of the patient's brain. Implanting the electrodes requires drilling burr holes within certain areas in the patient's skull to implant neuroprosthetic devices, e.g., electrodes, and an additional surgery to implant a stimulator. The stimulator is a pacemaker-like device implanted under the skin in the upper chest of a patient. The implanted electrodes deliver electrical stimuli, in the form of electrical impulses, to a region to activate voltage-sensitive ion channels in neuronal membranes. The electrical stimuli result in an induced action potential in the cell triggering the release of chemical signals in the form of neurotransmitter molecules.

The surgical procedures required for DBS are invasive and associated with a certain risk of complications, such as hemorrhage, brain stroke, infection, and hardware complications such as eroded wire, and foreign-body rejection. Due to the potential for complications, noncontact, non-traumatic control of neural excitation and inhibition has been of strong interest for both research and practice. Guided by the sensitivity of neurons to other physical stimuli, such as temperature, pressure, and tension stimuli, innovative non-contact methods utilizing acoustic and magnetic external stimuli have been of interest. However, acoustic and magnetic neuronal stimuli lack cellular specificity, i.e., the ability to target specific regions of cells with high spatial resolution, or target select types of cells.

Recently, developments have led to the emergence of a new field called optogenetics. Optogenetics utilizes gene transfection of light-sensitive ion channel proteins, known as opsins, to effect specific populations of neurons in the brain. Optogenetics allows for the control of neuronal activity using light with high temporal (millisecond scale) and spatial precision. Opsins are a family of membrane retinal containing proteins that transport ions across a membrane via multistep retinal chromophore isomerization in response to visible light. The transport of ions through the cell membrane results in either a depolarization, for an excitatory receptor response, or a hyperpolarization, for an inhibitory response in the cell. In other words, opsins, when activated by visible light, cause or inhibit an electrical response in the cells in which they reside. Certain microbial opsins, such as channelrhodopsin, are cation channels that excite neurons through membrane depolarization. Other opsins, such as proton-pump archaerhodopsin and chloride-pump halorhodopsin, mediate membrane hyperpolarization and thus inhibit neural activity.

Similar to electrical DBS, which uses microelectrodes, optogenetic brain stimulation still requires invasive procedures to implant optical hardware such as optical fibers or waveguides to deliver photons to target cells in the brain. Use of optical fibers, however, is limited by tissue absorbance and scattering which limits penetration depths to the millimeter scale using near-infrared (NIR) light within the NIR biological window. Additionally, apparatus are required to maintain the position of an optical fiber or waveguide to ensure the delivery of the photons to the desired location and to prevent movement of the optical fiber or waveguide to prevent potential damage to the brain cells and tissues. Due to the required invasive hardware and apparatus, optogenetics can cause complications such as the risk of trauma, infection, overheating by NIR light, and foreign-body reactions. Therefore, modern optogenetic brain stimulation faces key challenges for the delivery of NIR radiation through the cranium bones and into target areas of the brain.

### SUMMARY OF THE DISCLOSURE

A method for performing deep tissue stimulation using radiation-enabled optogenetics XR-optogenetics (XRO) includes introducing a plurality of nanoscintillators into a region with light sensitive cells, and targeting the nanoscintillators with a primary radiation to cause the nanoscintillators to emit optical energy. The light sensitive cells have an optically active wavelength range, and the emitted optical energy has a wavelength in the optically active wavelength range.

### BRIEF DESCRIPTION OF THE DRAWINGS

FIG. 1A is a diagram illustrating an electron at ground state in a three-state nanoscintillator.

FIG. 1B is a diagram illustrating radiation exciting an electron from the ground state to a second excited state in a three-state nanoscintillator.

FIG. 1C is a diagram illustrating emission of a photon and relaxation of an electron from a second excited state to a first excited state in a three-state nanoscintillator.

FIG. 1D is a diagram illustrating emission of a photon and relaxation of an electron from a first excited state to a ground state in a three-state nanoscintillator.

FIG. 2A illustrates an embodiment of a scenario for performing non-invasive XR-optogenetics deep brain stimulation.

FIG. 2B is a flow diagram of a method for performing non-invasive XR-optogenetics deep brain stimulation.

FIG. 3A is a flow diagram of a method for introducing a material that makes cells light sensitive to non-light sensitive cells, and performing XR-optogenetics deep brain stimulation on the optically sensitive cells.

FIG. 3B is a flow diagram of a method for introducing a material that makes cells light sensitive to sub-regions of tissue containing non-light sensitive cells, and performing XR-optogenetics deep brain stimulation on the sub-regions of tissue.

FIG. 4A is a transmission electron microscopy image of nanoscintillators for use in radiogenetic deep brain stimulation as described herein.

FIG. 4B is a high-resolution transmission electron microscopy image of nanoscintillators for use in XR-optogenetics deep brain stimulation as described herein.

FIG. 4C is a scanning transmission electron microscopy image of nanoparticles and corresponding energy dispersive X-ray spectroscopy data showing material densities and dopant levels in the nanoparticles.

FIG. 4D is a plot of the measured hydrodynamic diameter of the nanoparticles in a solution.

FIG. 5 is a plot showing an X-ray diffraction pattern of nanoparticles and a typical diffraction pattern of the nanoparticles.

FIG. 6 is a plot of the Fourier transform of the infrared transmittance spectrum of nanoparticles.

FIG. 7 is a plot of emitted optical luminescence of nanoparticles with and without the application of X-ray radiation.

FIG. 8 is an image of neuronal activity of tissues in the cerebral cortex using fluorescent tagging.

FIG. 9 is a plot of an evoked response using the methods disclosed.

FIGS. 10A and 10B are two-photon microscopy images of brain tissue showing channelrhodopsin expression.

FIGS. 11A-11D are fluorescence microscopy images showing channelrhodopsin expression and c-fos protein expression.

### DETAILED DESCRIPTION

The methods disclosed herein provide a means for performing non-invasive, non-traumatic deep brain stimulation (DBS). The methods described accomplish non-traumatic DBS by inducing action potentials in light sensitive cells through light delivery to the cells via stimulation of highly luminescent scintillators. The scintillators are introduced to a region or tissue(s) containing the light sensitive cells, and the scintillators are excited by providing high-energy photons or ionizing radiation to the scintillators. Radiation emitted by the scintillators during de-excitation may then be absorbed by opsins in light sensitive cells and cause the generation of action potentials in the optically sensitive cells. The methods described herein allow for the manipulation and control of neurons in neural networks in the intact brain, as well as manipulation of other types of optically sensitive cells.

For embodiments herein, the terms “primary excitation state”, and “secondary excitation state” may also be used to describe the energy states of a system. The term “primary excitation state” should be understood to be a non-ground state of a system, the transition to which is stimulated by a primary radiation provided by a primary radiation source, or due to relaxation from a higher energy state, as described below. Similarly, the term “secondary excitation state” should be understood to be a non-ground state of a system, the transition to which is induced by a primary radiation or excitation radiation, as also described below.

In electromagnetics, it is common to distinguish between a frequency, wavelength, energy, and color of electromagnetic radiation. Each of these four characteristics is related to the other three. For example, the wavelength, in nanometers (nm), and frequency, in hertz (Hz), for a specified electromagnetic radiation are inversely proportional to each other. Similarly, the energy, in electron-volts (eV) or joules (J), of electromagnetic radiation is proportional to the frequency of that radiation. Therefore, for a given radiation at a given frequency, there is a corresponding wavelength and energy.

The fourth of the aforementioned characteristics, color, typically represents a group or band of frequencies or wavelengths. For example, the color blue is commonly defined as electromagnetic radiation with a wavelength from 450 nm to 495 nm. This wavelength band also corresponds to frequencies from 606 THz to 668 THz, and energies of 2.5 to 2.75 eV. The color blue, then, is any radiation with one of those wavelengths, or radiation with multiple wavelengths in that band. Therefore, the term color may refer to one specific wavelength, or a band of wavelengths. Some areas of trade in electromagnetics prefer the use of one of the four terms to the others (e.g., color and wavelength are preferred when discussing optical filters, whereas frequency and energy are preferred when discussing optical excitation processes). Therefore, the four terms may be understood to be freely interchangeable in the following discussion of electromagnetic radiation and scintillators.

Additionally, as a person of ordinary skill in the art would understand, the terms excited state, excitation state, quantum state, and energy state can be interchangeable when describing the state of a system. Also, the states of a system may also be described as having or existing with a specific energy, E, associated with the state. Therefore, it should be understood that a state may be referred to as an energy state E, or a state with energy E interchangeably. As such, it should be understood that a label E may refer to the energy of a state and/or to the state itself. In photonics, and specifically when considering single photon emission, the terms emission time, relaxation time, relaxation rate, transmission rate, transition time, decay rate, and decay time are also understood to be related in most cases. In addition, a person of ordinary skill in the art would recognize that the terms excite, promote, or energize are often interchangeable when discussing the transition of a system from one energy level to another, higher, energy level, and similarly the terms de-excite, rest, and recombine may be used interchangeably when discussing the transition of a system from one energy level to another, lower, energy level.

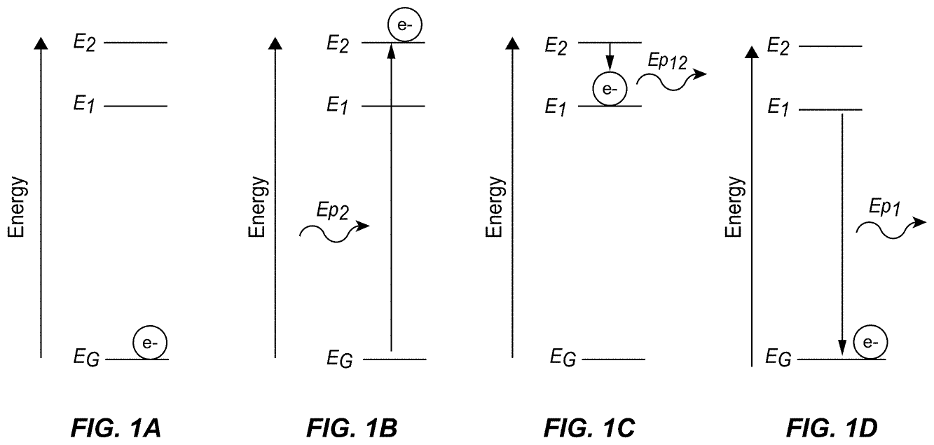

FIGS. 1A-1D show an electron, e-, in a scintillator, and three energy states: a ground energy state, E_{G}, a primary excited energy state, E₁, and a secondary excited energy state, E₂. The ground state E_{G} may be a valence band state (e.g., a heavy-hole, light-hole, or spin-orbit band or state). In FIGS. 1A-1D, the primary excited energy state is the lowest excited state or first excited energy state with energy E₁, and the secondary excited energy state is a second excited energy state with an energy E₂, greater than E₁ with both the primary and secondary bands being in the conduction band. As illustrated in FIG. 1A the electron typically exists in or occupies the ground energy state. The electron remains in the ground energy state until some form of excitation or perturbation changes the state of the electron in the scintillator. FIG. 1B illustrates an excitation energy provided by a photon with energy Eₚ₂. The photon provides energy to the electron exciting it to the second excited state, E₂. The energy gap between the ground state and the primary and secondary excited states may be determined by the material properties and dimensions of the scintillator, discussed further below. Once in the secondary excited state, the electron may couple to a vibrational mode, de-excite or relax down into the primary excited state, and emit a phonon, or the electron may relax into the primary excited state and emit a photon with energy Eₚ₁₂, as illustrated in FIG. 1C. Once in the primary excited state, the electron may further relax back into the ground state and emit a phonon, or emit a photon with energy Eₚ₁, as illustrated in FIG. 1D.

The emitted photons with energies Eₚ₁₂ and Eₚ₁ may have energies in the visible range, infrared range, ultraviolet range, or another energy range or band determined by the bandgaps between the ground state and the primary and secondary excited states, and between the primary excited state and the secondary excited state. The excitation energy Eₚ₂ may be provided by a photon with an energy equal to the band gap between the ground state and the secondary excited energy state, as illustrated in FIG. 1B, or the excitation energy may be an energy that is greater than the band gap between the ground state and the secondary excited energy state. As a person or ordinary skill in the art would recognize, the energy bands in many materials are not flat lines as illustrated in FIGS. 1A-1D, but are curved bands with energy peaks and valleys, and an electron may be excited into any part of a band and migrate along the band before relaxing into a lower energy state. Additionally, while FIGS. 1A-1D depict a system with only three energy states, systems may have any number of energy states with any number of ground or excited energy states.

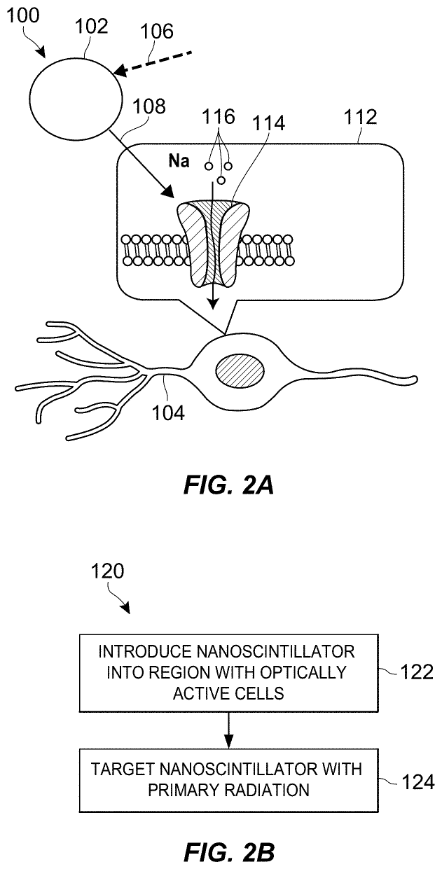

FIG. 2A illustrates an embodiment of a scenario 100 for performing non-invasive XR-optogenetics (XRO) DBS, as described herein, while FIG. 2B is a flow diagram of such an embodiment. Referring simultaneously to FIGS. 2A and 2B, the method 120 includes providing a nanoscintillator 102 (block 122) into a region containing an light sensitive cell 104 and targeting the nanoscintillator 102 with a primary radiation 106 (block 124). The primary radiation 106 is selected to excite the nanoscintillator 102, and may also be referred to herein as excitation radiation. As illustrated previously in FIGS. 1A-1D, excitation radiation excites an electron in the nanoscintillator 102, and de-excitation of the electron results in emission of optical energy 108. The optical energy 108 may then activate the light sensitive cell 104, causing an electrical potential, known as an evoked potential or evoked response, in the light sensitive cell 104. The inset 112 shown in FIG. 2A shows the optical energy 108 incident on a light-gated ion channel 114 in the cell membrane of the light sensitive cell 104, which opens the light-gated ion channel 114, allowing ions 116 to enter the light sensitive cell 104 causing the evoked response, which will be discussed further below.

Many nanoscale materials and components constrain electrons, or other charge carriers, in one or more spatial dimension. This spatial restriction is known as quantum confinement. The effects of quantum confinement cause charged carriers in a quantum well to occupy only discrete energy levels of the quantum well, as illustrated in FIGS. 1A-1D. The energy bands or energy states are determined by the quantum well material dimensions (i.e., length, width, height, radius, semi-major axis, semi-minor axis, and/or circumference), electromagnetic properties, the specific materials and dopants of the nanoscale material or device, and the number of atomic layers. Additionally, the energy bands and energy subbands determine the radiative wavelengths at which the quantum well is optically active (i.e., optically absorptive, transmissive, and emissive properties of the quantum well). Therefore, depending on the desired wavelengths of optical activity, the nanoscintillator 102 may be a nanotube, a nanorod, a nanotip, a wire, a pillar, a disc, a sheet or ribbon, a quantum dot, a quantum well, a nanocluster, a nanopowder, a nanocrystal, a nanoparticle, or any combination thereof.

In embodiments, the nanoscintillator 102 may be a 1 D, 2D, or 3D material that emits optical photons when excited by ionizing radiation, including high-Z materials (e.g. Ti, Cr, V, Fe), nanophosphors, lanthanide-doped tungstate, an X-ray excited optical luminescent material, silica, gold nanoparticles, a nanophosphor, mesoporous silicon dioxide or silicate, a perovskite, a quantum dot, an organo-metallic cluster (such as metal-organic frame works MOFs, coordination and nanoparticle-doped polymers etc.) or any combination thereof.

In embodiments, the nanoscintillator 102 may be a nanoparticle, fine particle, or an ultrafine particle with at least one dimension (i.e., length, width, height, radius, semi-major axis, semi-minor axis, and/or circumference) of the nanoparticle being between 1 and 10 nanometers, 1 and 100 nanometers, 1 and 500 nanometers, 1 and 2500 nanometers, 100 and 500 nanometers, 100 and 1000 nanometers, 500 and 1000 nanometers, 250 and 500 nanometers, 250 and 1000 nanometers, 500 and 1500 nanometers, 500 and 2500 nanometers, 1000 and 1500 nanometers, 1000 and 2500 nanometers, 1500 and 2500 nanometers, or 2000 and 2500 nanometers. In embodiments, the nanoscintillator 102 may be suspended in a solution and introduced to tissue in amounts on the order of hundreds of nanoliters, microliters, tens of microliters, hundreds of microliters, milliliters, or centiliters. Additionally, the concentration of the nanoscintillators in the suspension may be between less than 1 mg/ml, on the order of milligrams per ml, on the order of tens of mg/ml, or on the order of hundreds of mg/ml.

In embodiments, the light sensitive cell 104 may be a cell with light-gated ion channels, as illustrated in FIG. 2A. Gated ion channels are proteins that provide a selective pathway for charged ions (e.g., sodium, potassium, calcium, chloride, etc.) to pass through a cell membrane. The gated ion channel may open or close based on the presence of a pressure, a temperature, a voltage, a particular neurotransmitter or neuromodulator, or other factor. Light-gated ion-channels are gated based on the presence of an electromagnetic field, and more specifically different light-gated ion channels open or close based on the presence of specific wavelengths of electromagnetic energy, referred to herein as the optically active wavelength range of the ion-channel or of a light sensitive cell. It is possible to express ion channels sensitive to different wavelengths of light in different target cells of the brain. Being activated by light, channelrhodopsins allow cations to flow into a neuron, thus depolarizing the neural membrane and triggering an action potential. In embodiments, light-sensitive chloride channels may be used as optogenetic inhibitors, i.e. light of particular wavelengths may cause hyperpolarization of a neural membrane and block the generation of action potentials. The passage of the ions through the ion channel may cause an evoked response in a cell, and restricting the passage of ions may repress an action potential in a cell.

Different ion channels allow ions to move into or out of neurons. In neurons, ion channels promote neurotransmission by altering the polarization of the cellular membrane. Thus, cations flow into the postsynaptic terminal which may result in an excitatory postsynaptic potential (EPSP). Alternatively, the flow of anions into the postsynaptic terminal may cause an inhibitory postsynaptic potential (IPSP). An EPSP increases the chance of a neuron to generate an action potential (AP) and propagate a neuronal signal, and an IPSP decreases the chance of a neuron to generate an action potential. In neurons, ion channels can be classified by two groups: voltage-gated channels and ligand-gated channels. Voltage-gated channels are activated by changes in the electrical potential across a membrane. Ligand-gated channels are activated by the binding of a specific ligand (e.g., a neurotransmitter) to the ion channel. Typically, different types of channels selectively control the flow of one ion type, such as sodium, calcium, potassium, or chloride, across a membrane. Light-controllable channels in the neural membrane allow for the excitation or inhibition (depending on the channel type) of action potentials by light in neurons.

In embodiments, the light sensitive cell may be a cell with an opsin as a light-gated ion channel, e.g., channelrhodopsin with an optically sensitive region in the red spectrum, around 615 nm. Therefore, the wavelengths of a primary, or excitation radiation may be around 615 nm to evoke a response in the light sensitive cell. In embodiments, the nanoscintillator may be chosen to be lanthanide-doped tungstate phosphor particles, with a grain size of 100 nm. The emission spectrum of lanthanide-doped tungstate phosphor particles is tunable from around 590 to 630 nm. Therefore, by tuning the doping and spatial dimensions of the lanthanide-doped tungstate phosphor particles, an emission spectrum that matches, or emits radiation in, the optically active wavelength range of the light sensitive cell may be achieved. As one of ordinary skill in the art would recognize, an electronic band structure is determined by the material makeup and dimensions of a nanoscintillator, and therefore the absorption and emission spectra of a nanoscintillator (i.e., around 615 nm).

In embodiments, the optically active wavelength range may be infrared wavelengths, visible wavelengths, ultraviolet wavelengths, 200 to 500 nm, 400 to 700 nm, 600 to 900 nm, 200 to 700 nm, 200 to 900 nm, shorter than 1000 nm, longer than 200 nm, or any light sensitive region of a light-gated ion channel in an light sensitive cell. In embodiments, the primary radiation, or excitation radiation, may be X-ray radiation, gamma radiation, ultraviolet radiation, beta radiation, Cherenkov radiation, or another type of radiation with energy high enough to excite a nanoscintillator.

In embodiments, the light sensitive cell 104 may be a neuron in synaptic communication with other cells and the evoked response may result in the firing of action potentials in other cells causing activation of regions of tissue. In embodiments, the light sensitive cell 104 may be a neuron and evoking a potential in the neuron may cause other neurons to fire, thereby allowing for stimulation of parts of nerve tissues, or parts of the brain. Controlling the neural activity may be implemented in research and/or treatment for dystonia, epilepsy, essential tremors, obsessive-compulsive disorder, Parkinson's disease, addiction, chronic pain, cluster headaches, dementia, depression, Huntington's disease, multiple sclerosis stroke recovery, Tourette syndrome, forms of blindness, spinal cord restoration, functional brain mapping, and traumatic brain injuries among other diseases and conditions that effect neuronal tissues. Additionally, the light sensitive cell may be a motor neuron, interneuron, sensory neurons, a glial cell, or another type of cell.

In embodiments, the light sensitive cell 104 may be a smooth muscle cell such as a myocardiocyte. In embodiments in which the light sensitive cells are myocardiocytes, evoking a potential in the myocardiocytes may be implemented in therapies for cardiac dysrhythmia, bradychardia, tachycardia, flutter, fibrillation, and premature contraction among other types of cardiac conditions. In embodiments, the light sensitive cell 104 may be a skeletal muscle cell, a motoneuron, or a myocyte. The methods disclosed may be employed in therapies that require controlling contractions of muscles, or for stimulating muscle cells.

In embodiments, the light sensitive cell 104 may be a motor neuron, sensory neuron, interneuron, and/or any neuron in the brain, spinal cord, or peripheral nerve system. In embodiments the light sensitive cell 104 may be a glial cell such as an oligodendrocyte, an astrocyte, a microglial cell, a skeletal muscle cell, a smooth muscle cell, or an epithelial cell In embodiments, the light sensitive cell 104 may be any channel-sensitive cell with a light-gated ion channel. Further, while the light sensitive cell 104 has been described in the above embodiments as a neuron or a myocardiocyte, in embodiments, the light sensitive cell 104 may be any cell able to generate and/or receive an electrical signal and the light sensitive cell 104 may be in electrical communication with other cells, or in biological or chemical communication with other cells (e.g., by expression and/or reception of neurotransmitters, neuromodulators, a signal peptide, a signal molecule, a hormone, a protein, reception at a receptor protein, etc.).

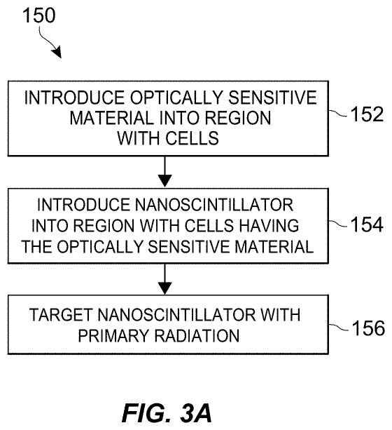

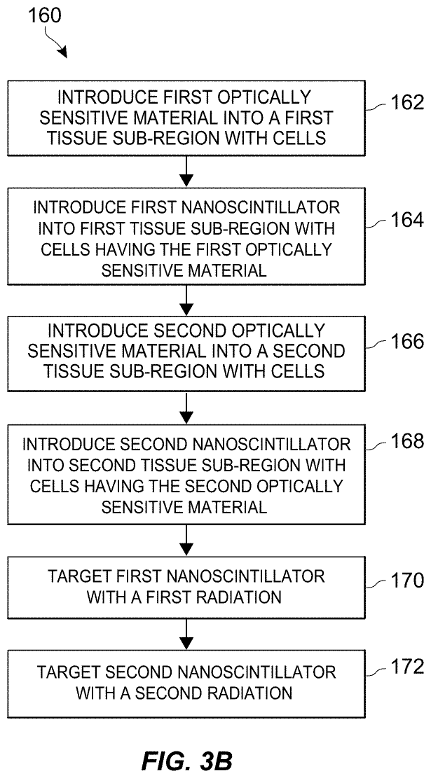

In embodiments of a method for performing XRO DBS, it may be desirable to perform XRO DBS in tissues or a region of cells with non-light sensitive cells. Therefore, an light sensitive material, or a material that makes cells light sensitive, may be introduced to a region with cells to create the light sensitive cells. FIG. 3A is a flow diagram of a method 150 for introducing an optically sensitive material to non-light sensitive cells, and performing XRO DBS on the light sensitive cells. The method 150 includes introducing, into a region with cells, an optically active material (block 152). In embodiments, introducing the optically active material may include introducing a viral vector to a region of cells. The viral vector may contain an optogenetic actuator gene that, when introduced to the cells, causes the cells to express the optogenetic actuator gene and produce microbial opsins. After the cell begins to produce the microbial opsins, the cells are light sensitive. The optically active material may be introduced by injection, ingestion, through inhalation, by a nasal spray, through a soft membrane, through an oral mucosa, through a gastric mucosa, or through another mucous membrane. In descriptions herein of providing a light sensitive material or optically active material to a region, some embodiments may employ providing a material (e.g., a virus, primer, viral vector, etc.) that makes cells light sensitive.

The method 150 of FIG. 3A further includes introducing a nanoscintillator into the region with the cells having the optically sensitive channels, i.e., the light sensitive cells (block 154). The nanoscintillator may be introduced by injection, ingestion, through inhalation, by a nasal spray, through a soft membrane, through an oral mucosa, through a gastric mucosa, or through another mucous membrane, through injection (e.g., Cisterna Magna injection), or via focused reversible blood-brain barrier (BBB) opening (e.g. using sonoporation and “micro-bubbles”). The nanoscitillator may be also delivered to the target area of the brain or any other part of an organ, organism, or tissue in the form of molecule-conjugated nanostructures or combined with liposomes, vesicles, magnetosomes, or by any combination of these methods. Targeting molecules (e.g., ligands), which serve the function of ensuring the nanoscintillators target particular cells of interest, may include antibodies, single chain variable fragments (scFvs), BBB-penetrating peptides, aptamers, hyaluronic acid, via magnetic targeting or by the combination of these methods. In embodiments, it may be desirable for the distance between the nanoscintillator and the light-sensitive cell to be 200 to 300 microns, 200 microns or less, 300 microns or less, or 500 microns or less. The distance between a nanoparticle and a light sensitive cell may depend on the wavelength of emitted radiation from the nanoscintillator, e.g., NIR light penetrates brain tissue on the order of millimeters while green and blue light only penetrates brain tissue on the order of tens of microns. Additionally, the desired distance of the nanoscintillator from the light-sensitive cell may depend on the quantum efficiency of the nanoscintillator, e.g., it may be desirable for nanoscintillators with lower quantum efficiencies to be closer to the light-sensitive cell to increase the chance of successful light delivery to the light-sensitive cell.

In embodiments, the optically active material and the nanoscintillator may be introduced simultaneously by any of the means listed herein. Introducing the gene or genes which make cells express light-sensitive peptides and nanoscintillator simultaneously may provide some control over ensuring that the nanoscintillator and gene or genes which make cells express light-sensitive peptides are introduced to the same region, which may increase the efficiency of XRO DBS as described herein. In embodiments, the nanoscintillator and/or the gene or material which makes cells express light-sensitive peptides may be introduced within a sub region of tissue, or the nanoscintillator and/or the genes which make cells express light-sensitive peptides may be introduced over an entire tissue or tissue sample. In embodiments that employ a viral vector to express light sensitive peptides in a region of cells, the viral vector may target specific types of cells. The viral vector may be introduced to a large region of cells or an entire tissue sample or organ, and the viral vector may migrate to the specific cells to be provided with XRO DBS. In embodiments, a viral vector may be provided to an entire tissue sample, and nanoscintillators introduced to sub-regions of the tissue for DBS of specific regions of a tissue sample, described further in reference to FIG. 3B. In embodiments, the material which makes cells express light-sensitive peptides may be suspended in a solution and introduced to tissue in amounts on the order of hundreds of nanoliters, microliters, tens of microliters, hundreds of microliters, milliliters, or centiliters. Additionally, the concentration of the material which makes cells express light-sensitive peptides in the suspension may be hundreds of mega-genome copies per milliliter (MGC/ml), between one and two tera-genome copies per milliliter (TGC/ml), between one and ten TGC/ml, tens of TGC/ml, or hundreds of TGC/ml.

The method 150 of FIG. 3A further includes targeting the nanoscintillator with primary radiation (block 156), the primary radiation configured to excite the nanoscintillator. The nanoscintillator may then emit a photon in an optically active wavelength range of the light sensitive cells to evoke an action potential in the light sensitive cells. The primary radiation may be any type of radiation as previous described, the primary radiation having an energy capable of exciting the nanoscintillator into an excited state.

In embodiments that employ opsins as the optically active material, the light sensitive peptide may be a channelrhodopsin, bacteriorhodopsin, xanthorhodopsin, halorhodopsin, archaerhodopsin, L-opsin, M-opsin, S-opsin, R-opsin, pinopsin, parapinopsin, parietopsin, encephalopsin, RGR-opsin, neuropsin, or another opsin. Additionally, the optically active material may be an artificial light-sensitive ion channel, artificial ion pump, a pore, an ionopore, or any light sensitive or radiosensitive peptide.

In embodiments, nanoscintillators and/or optically active materials may be introduced to sub-regions of a tissue (e.g., different regions in a brain). Additionally, different types of nanoscintillators and/or optically active materials may be introduced in different sub-regions. Different nanoscintillators and/or optically active materials may allow for the separate probing of distinct or overlapping regions of interest. For example, a first excitation energy may be applied to a first sub-region of tissue to probe the first sub-region, and a second excitation energy may be applied to a second sub-region of tissue to probe the second sub-region. Such an embodiment may allow for different sub-regions of the same tissue, or organ, to be stimulated at the same time, or stimulated independently as desired. Probing sub-regions of tissue may be useful in treatments requiring simultaneous, or sequential, stimulation of different regions of tissue. As but one example, the method may be used to probe regions of the brain during brain surgery in order to pinpoint a desired location of surgical treatment. By probing specific areas of the brain independently—for example by applying radiation having an energy tuned to the nanoscintillators and/or optically active materials specific to that region—a region having a specific function may be located.

In embodiments, an optically active material may be introduced to an entire organ or large region of tissue (e.g., an entire brain lobe or an entire brain) and nanoscintillators may be introduced into various sub-regions of the tissue. Different types of nanoscintillators may be excited by different frequencies of applied primary radiation. Therefore, different portions or sub-regions of a tissue may be probed by applying different frequencies of primary radiation to the tissue. In embodiments, various optically active materials having different optically active wavelength ranges may be introduced into different sub-regions of a tissue. Various nanoscintillators that emit optical radiation at corresponding optical wavelength ranges may be introduced to the regions with the optically active material having the corresponding optically active wavelength range. Such embodiments may be used to probe various sub-regions of a tissue (e.g., different regions in a brain) independently as desired for a treatment or for brain mapping.

FIG. 3B is a flow diagram of an embodiment of a method 160 for performing XRO DBS in sub-regions of tissue. The method 160 includes introducing a first optically sensitive material into a first tissue sub-region (e.g., a sub-region of a brain, heart, or other organ) that has non-light sensitive cells (block 162). The introduction of the first optically sensitive material causes the non-optically sensitive cells to be optically sensitive to a first optically active wavelength band, or first optically sensitive region. A first nanoscintillator is introduced into the first tissue sub-region containing the cells having the first optically active material (block 164). The introduction of the first genes making cells express light-sensitive peptides and the first nanoscintillators may be performed independently or simultaneously by any of the ways previously described herein. The method further includes introducing a second optically sensitive material into a second tissue sub-region with cells (block 166) and introducing a second nanoscintillator into the second tissue sub-region that contains the cells having the second optically sensitive material (block 168). The introduction of the second optically sensitive material causes the non-light sensitive cells to be light sensitive to a second optically active wavelength band, or second optically active region. Similarly, the introduction of the second optically active sensitive material and the second nanoscintillators may be performed independently or simultaneously by any of the ways previously described herein.

The method 160 of FIG. 3B further includes targeting the first nanoscintillator with first radiation (block 170), the first radiation selected to excite the first nanoscintillator. The first nanoscintillator may then emit a photon in the first optically active wavelength range of the first optically active material to evoke an action potential in the first light sensitive cells. The second nanoscintillator may then be targeted with a second radiation (block 172), the second radiation selected to excite the second nanoscintillator. The second nanoscintillator may then emit a photon in the second optically active wavelength range of the second optically active sensitive material to evoke an action potential in the second light sensitive cells. The first and second radiations may be any type of radiation as previous described, the first and second radiations having energies capable of exciting the first and second nanoscintillators, respectively, into excited states.

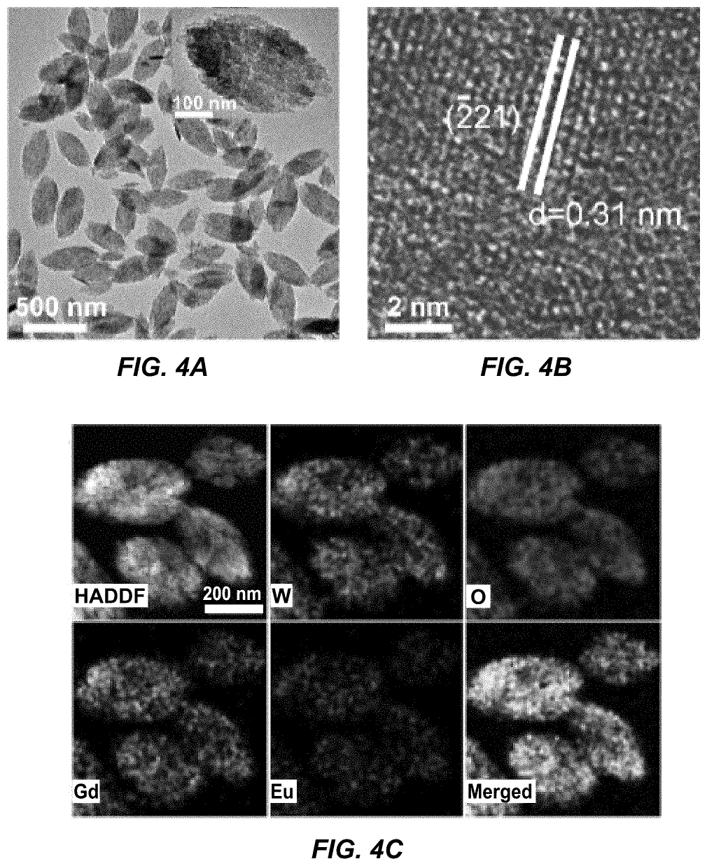

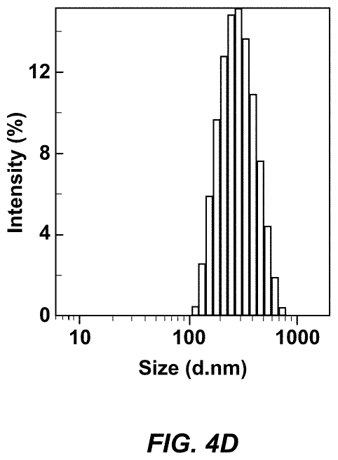

FIGS. 4A and 4B are a transmission electron microscopy (TEM) image and a high-resolution TEM image, respectively, of nanoscintillators for use in XRO DBS as described herein. The nanoscintillators of FIGS. 4A and 4B are lanthanide-doped tungstate nanoparticles with composition Gd₂(WO₄)₃: 10% Eu, referred to herein as GdWEu nanoparticles. The TEM images revealed that the nanoparticles display a spindle-like shape with an average equatorial diameter of ˜160 nm with a length of ˜360 nm. The nanoparticles have tunable scintillation capabilities that allow for radiation emission with wavelengths from around 590 to 630 nm under X-ray excitation. FIG. 4C is a scanning TEM of the GdWEu nanoparticles and corresponding energy dispersive X-ray spectroscopy data confirming the desired material densities, or dopant levels, in the GdWEu nanoparticles. The scale bar for all panels in FIG. 4C is 200 nm. FIG. 4D is a plot of the measured hydrodynamic diameter of the GdWEu nanoparticles in a solution. The particles in FIGS. 4A-4D were developed for demonstration of the methods for XRO DBS as described herein.

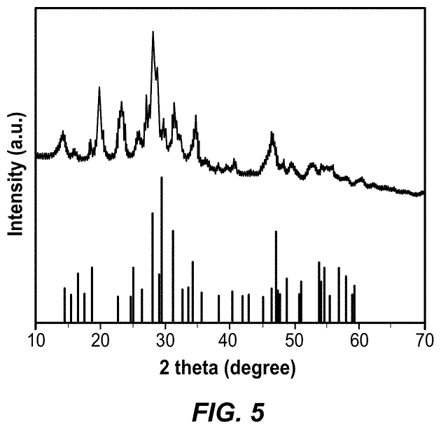

FIG. 5 is a plot showing an X-ray diffraction pattern of the GdWEu nanoparticles (top line) compared to a typical diffraction pattern of GdWEu nanoparticles (bottom peaks) based on data from the International Centre for Diffraction Data. The measured diffraction pattern matches closely with the peaks from the data provided by the International Centre for Diffraction Data.

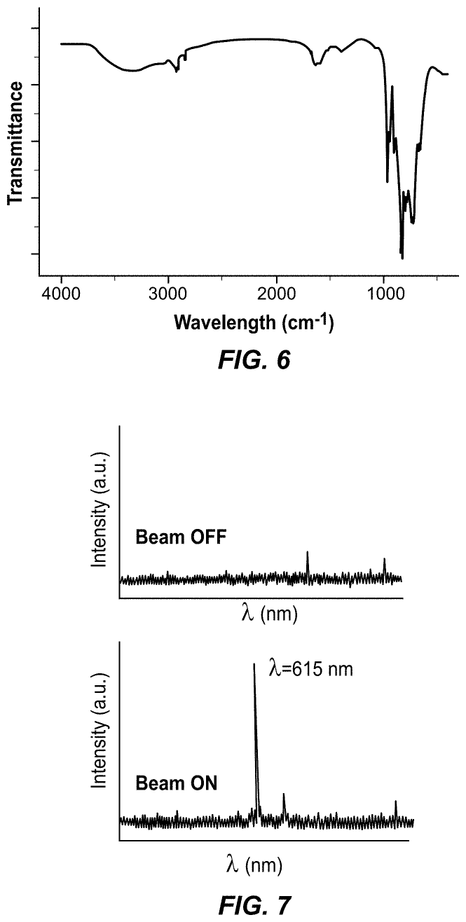

FIG. 6 is a plot of the Fourier transform of the infrared transmittance spectrum of the GdWEu nanoparticles. The broad band between 3600 and 3000 cm⁻¹ corresponds to an O—H stretching vibration arising from surface hydroxyl groups of absorbed water and ethylene glycol. The peaks at 2930 and 2852 cm⁻¹ correspond to C—H stretching. The peaks at 1633 cm⁻¹ and 1000 cm⁻¹ are assigned to O—H bending and C—O stretching, and the peak at 730 cm⁻¹ is due to an in-plane rocking vibration of —CH₂—. The absorption of ethylene groups by the GdWEu nanoparticles could be due to nonspecific absorption or through formation of M-OCH2CH2OH bonds (M refers to the metal ions on the surface of the crystal) during the nanoparticle crystal growth.

FIG. 7 is a plot of the emitted optical luminescence of the GdWEu nanoparticles without the application of X-ray radiation (top plot) and with the application of X-ray radiation (bottom plot). The top plot in FIG. 7 shows that the GdWEu nanoparticles do not luminesce, or emit radiation, without the presence of X-ray radiation. The bottom plot in FIG. 7 shows that the GdWEu nanoparticles emit radiation at a wavelength of 617 nm under applied X-ray radiation. The phenomena of generating optical luminescence from a particle using X-ray excitations is known as X-ray excited optical luminescence (XEOL), or Radioluminescence (RL), and the GdWEu nanoparticles may be considered to part of a class of XEOL particles. The X-ray radiation described in reference to FIG. 7 may be considered to be the primary radiation, or excitation radiation as discussed in other embodiments herein.

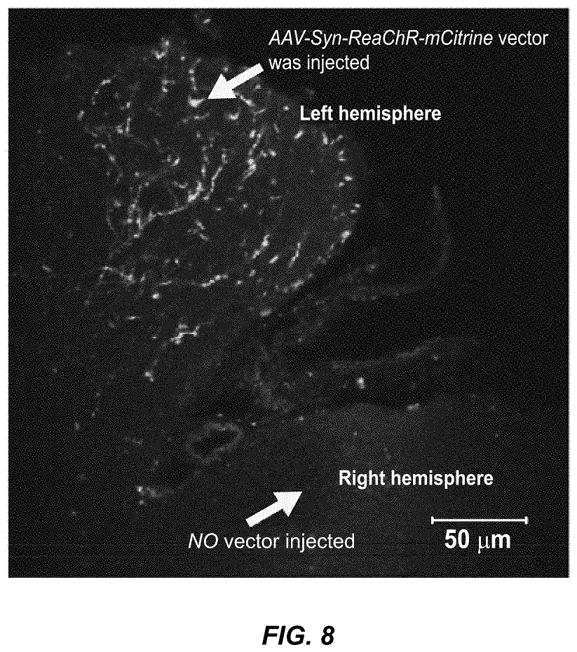

FIG. 8 is a grayscale image of neuronal activity of tissues in the cerebral cortex using fluorescent tagging. The left hemisphere of the cerebral cortex shown in FIG. 8 contains light sensitive cells containing channelrhodopsin, and nanoscintillators in the form of GdWEu nanoparticles. The right hemisphere has been provided with neither the channelrhodopsin or GdWEu nanoparticles. In FIG. 8, X-ray radiation is being provided to both the right and left hemispheres of the cerebral cortex. The dark region in the right hemisphere in FIG. 8 shows that there is no light-sensitive channelrhodopsin expressed in the right hemisphere. Alternatively, the bright spots and webs visible in the left hemisphere show that there is expression of channelrhodopsin, and thus, neurons in this part of the brain are light sensitive. The contrast of the neuronal action in the right and left hemispheres of FIG. 8, demonstrates one embodiment of the methods for performing XRO DBS described herein.

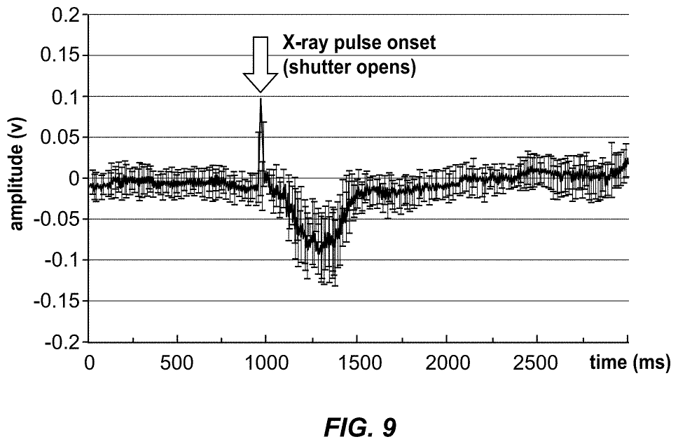

FIG. 9 is a plot of an evoked response using the methods disclosed. Evoked potentials (EP), or evoked responses, measure the electrophysiological responses of the brain to a simultaneous activation of many target neurons. The region of the targeted neurons for the EP shown in FIG. 9 is the primary motor cortex (M1). The method of EP involves averaging electroencephalography (EEG) activity time-locked to the introduction or presentation of an X-ray irradiation to the tissue region. The light sensitive cells containing channelrhodopsin were provided GdWEu nanoparticles which were injected into the region containing the optically sensitive cells, the nanoparticles acting as nanoscintillators according to the methods described herein. An X-ray pulse is applied to the light sensitive cell and the evoked potential is observed in FIG. 9 at 1000 ms. The X-ray pulse duration was around two to three seconds long and the evoked action potential lasted about 0.5 s. Commercial or custom-made shutters for the X-ray radiation may allow for temporal control of X-ray application potentials on the millisecond time scale. Short-term but repeatable application of X-ray radiation is physiologically more effective than continuous application, and allows for a decrease of the intensity of the applied radiation. Additionally, fine spatial resolution and control of the X-ray radiation may be implemented to provide XRO DBS to multiple regions of targeted tissues simultaneously, or sequentially, depending on a medical condition and the desired treatment. For example, as should be apparent, such spatial resolution may be achieved by tightly controlling the regions of delivery of the genes making cells express light-sensitive peptides to areas with neurons or groups of neuron. Alternatively, the fine spatial resolution may be achieved by tightly controlling the regions of delivery of the nanomaterials or nanoscintillators. In embodiments, tightly controlling the region of introduction of both the genes making cells express light-sensitive peptides and the nanomaterials may be implemented to achieve a desired XRO DBS spatial resolution.

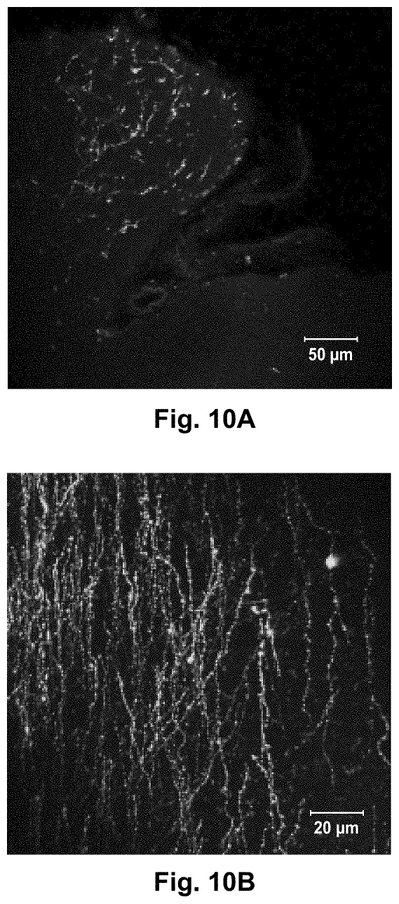

The GdWEu nanoparticles employed in the experiments described herein were found to be biologically inert, stable, and non-toxic. No immune response, inflammation, or other side effects were detected. FIGS. 10A and 10B are grayscale two-photon microscopy images of brain tissue showing channelrhodopsin expression (bright lines/regions). The images shown in FIGS. 10A and 10B were taken near the injection site of the nanoparticles and channelrhodopsin expression genes. No evidence of pathological processes were observed in the brain from nanoparticle injection to the end of the experiments (from three to five months). Additionally, all animals maintained good health conditions, had a normal body weight and behavior, and showed no visible signs of pain or stress.

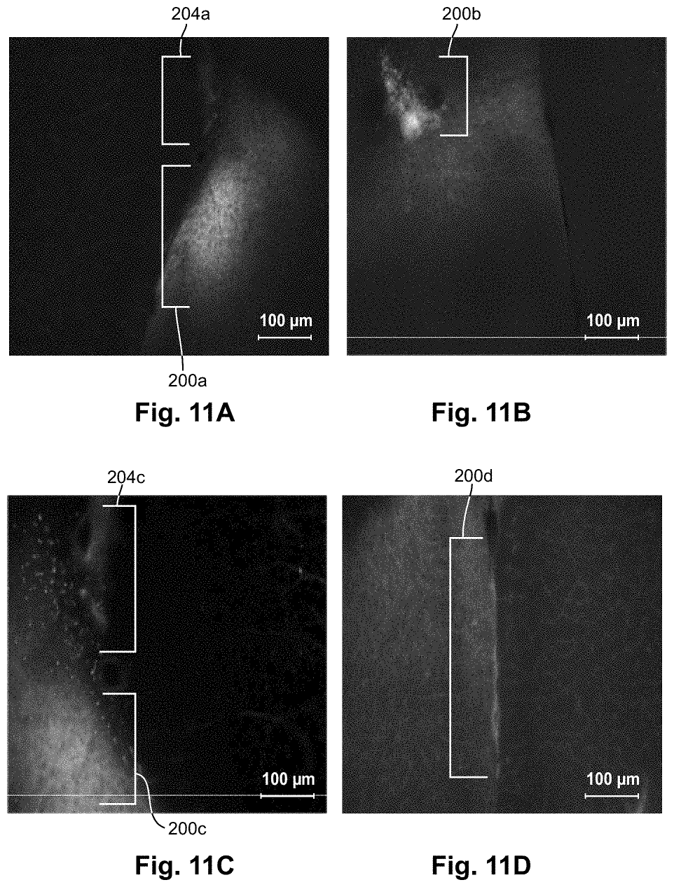

FIGS. 11A-11D are grayscale fluorescence microscopy images showing channelrhodopsin expression (originally shown as green regions in color images, and denoted in FIGS. 11A-11D by regions 200a through 200d) and c-fos protein expression (originally shown as red regions in color images, and denoted in FIGS. 11A and 110 by regions 204a and 204c), indicating neuronal activity. At the final stages of the experiments, the animals were euthanized and the c-fos expression method was used as a marker to measure neuronal activity (i.e., c-fos is expressed when neurons fire action potentials). FIGS. 11A and 110 demonstrate that the targeted neurons in the injection cite stayed alive until the animal was euthanized. The methods and materials described herein provide a means for non-invasive, non-traumatic methods for DBS unattainable by other technologies.

The following list of aspects reflects a variety of the embodiments explicitly contemplated by the present disclosure. Those of ordinary skill in the art will readily appreciate that the aspects below are neither limiting of the embodiments disclosed herein, nor exhaustive of all of the embodiments conceivable from the disclosure above, but are instead meant to be exemplary in nature.

1. A method for performing deep tissue stimulation using XR-optogenetics, the method comprising: introducing a plurality of nanoscintillators into a region with light sensitive cells, the light sensitive cells having an optically active wavelength range; and targeting the nanoscintillators with a primary radiation to cause the nanoscintillators to emit optical energy at a wavelength in the optically active wavelength range.

2. The method of aspect 1, wherein the plurality of nanoscintillators comprises a nanotube, a nanorod, or nanotip, a wire, a pillar, a disc, a sheet or ribbon, a quantum dot, a quantum well, a nanocluster, a nanopowder, a nanocrystal, a nanoparticle, an nanocluster or any combination thereof.

3. The method of either aspect 1 or aspect 2, wherein the plurality of nanoscintillators comprises a monolayer material, a phosphor, lanthanide-doped tungstate, an X-ray excited optical luminescence material, silica, silicon dioxide, gold nanoparticles or any combination thereof.

4. The method of any one of aspects 1 to 3, wherein the plurality of nanoscintillators comprises nanoparticles having at least one dimension between 1 and 10 nanometers long.

5. The method of any one of aspects 1 to 3, wherein the plurality of nanoscintillators comprises nanoparticles having at least one dimension between 1 and 100 nanometers long.

6. The method of any one of aspects 1 to 3, wherein the plurality of nanoscintillators comprises nanoparticles having at least one dimension between 1 and 500 nanometers long.

7. The method of any one of aspects 1 to 3, wherein the plurality of nanoscintillators comprises nanoparticles having at least one dimension between 1 and 2500 nanometers long.

8. The method of any one of aspects 1 to 7, wherein the optically active wavelength range comprises infrared wavelengths, visible wavelengths, ultraviolet wavelengths, or any combination thereof.

9. The method of any one of aspects 1 to 8, wherein the primary radiation is X-ray radiation.

10. The method of any one of aspects 1 to 8, wherein the primary radiation is gamma radiation.

11. The method of any one of aspects 1 to 10, wherein the light sensitive cells comprise neurons, myocardiocytes, spinal interneurons, nerve cells, motoneurons, myocytes, glia, a channel sensitive cell, skeletal muscle cells, smooth muscle cells, or any combination thereof.

12. The method of any one of aspects 1 to 11, wherein introducing a plurality of nanoscintillators into a region comprises injecting the nanoscintillators into the region.

13. The method of any one of aspects 1 to 11, wherein introducing a plurality of nanoscintillators into a region comprises applying a dosage of a nasal spray to a patient.

14. The method of any one of aspects 1 to 13, further comprising introducing an optically sensitive material to a region with cells to create the light sensitive cells.

15. The method of aspects 14, wherein introducing the optically sensitive material to a region with cells comprises introducing a viral vector into a region within a tissue sample.

16. The method of aspects 14, wherein introducing the optically sensitive material to a region with cells comprises introducing a viral vector to an entire tissue sample.

17. The method of any one of aspects 14 to 16, wherein introducing the plurality of nanoscintillators and introducing the optically sensitive material are performed simultaneously by injecting the nanoscintillators and the optically sensitive material into the same region with cells.

18. The method of any one of aspects 1 to 17, wherein the light sensitive cells comprise cells containing an opsin.

19. The method of any one of aspects 1 to 18, further comprising tuning the wavelength of the optical energy emitted by the nanoscintillators.

20. The method of aspects 19, wherein tuning the wavelength of the optical energy emitted by the nanoscintillators comprises doping of the scintillators.

21. The method of either aspects 19 or claim 20, wherein tuning the wavelength of the optical energy emitted by the nanoscintillators comprises tuning the dimensions of the nanoscintillators.

22. The method of any one of aspects 19 to 21, wherein tuning the wavelength of the optical energy emitted by the nanoscintillators comprises tuning the electronic band structure of the nanoscintillators.

23. The method of any one of aspects 1 to 22, further comprising tuning the decay rate of excited energy states in the nanoscintillators electronic band structure.

24. The method of aspect 23, wherein tuning the decay rate of excited energy states in the nanoscintillators electronic band structure comprises doping of the nanoscintillators.

25. The method of aspect 23, wherein tuning the decay rate of excited energy states in the electronic band structure of the nanoscintillators comprises tuning the dimensions of the nanoscintillators.

26. The method of any of aspects 1 to 25, wherein introducing a plurality of nanoscintillators into a region with light sensitive cells comprises introducing a first plurality of nanoscintillators into a first tissue sub-region with light sensitive cells, and introducing a second plurality of nanoscintillators into a second tissue sub-region with light sensitive cells.

27. The method of aspect 26, further comprising directing primary radiation of a first energy at the first plurality of nanoscintillators in the first sub-region and directing primary radiation of a second energy at the second plurality of nanoscintillators in the second-sub region.

28. The method of any of aspects 1 to 27, further comprising introducing a first optically sensitive material into a first tissue sub-region, and introducing a second optically sensitive material into a second tissue sub-region.

1. A method for performing deep tissue stimulation using XR-optogenetics, the method comprising:
introducing a plurality of nanoscintillators into a region with light sensitive cells, the light sensitive cells having an optically active wavelength range; and
targeting the nanoscintillators with a primary radiation to cause the nanoscintillators to emit optical energy at a wavelength in the optically active wavelength range.

2. The method of claim 1, wherein the plurality of nanoscintillators comprises a nanotube, a nanorod, a quantum dot, a quantum well, a nanocluster, a nanopowder, a nanocrystal, or any combination thereof.

3. The method of claim 1, wherein the plurality of nanoscintillators comprises a monolayer material, a phosphor, lanthanide-doped tungstate, an X-ray excited optical luminescence material, silicon dioxide, a perovskite, gold nanoparticles or any combination thereof.

4. The method of claim 1, wherein the plurality of nanoscintillators comprises nanoparticles having at least one dimension between 1 and 2500 nanometers long.

5. The method of claim 1, wherein the optically active wavelength range comprises infrared wavelengths, visible wavelengths, ultraviolet wavelengths, or any combination thereof.

6. The method of claim 1, wherein the primary radiation is X-ray radiation.

7. The method of claim 1, wherein the primary radiation is gamma radiation.

8. The method of claim 1, wherein the primary radiation is Cherenkov radiation.

9. The method of claim 1, wherein the light sensitive cells comprise neurons, myocardiocytes, spinal interneurons, nerve cells, motoneurons, myocytes, glia, a channel sensitive cell, skeletal muscle cells, smooth muscle cells, epithelial cells or any combination thereof.

10. The method of claim 1, wherein introducing a plurality of nanoscintillators into a region comprises injecting the nanoscintillators into the region.

11. The method of claim 1, wherein introducing a plurality of nanoscintillators into a region comprises applying a dosage of a nasal spray to a patient.

12. The method of claim 1, further comprising introducing a material that makes cells light sensitive to a region with cells.

13. The method of claim 12, wherein introducing the material that makes cells light sensitive to a region with cells comprises introducing a viral vector into a region within a tissue sample.

14. The method of claim 12, wherein introducing the material that makes cells light sensitive to a region with cells comprises introducing a viral vector to an entire tissue sample.

15. The method of claim 12, wherein introducing the plurality of nanoscintillators and introducing the material that makes cells light sensitive are performed simultaneously by injecting the nanoscintillators and the material that makes cells light sensitive into the same region with cells.

16. The method of claim 1, further comprising tuning the wavelength of the optical energy emitted by the nanoscintillators.

17. The method of claim 16, wherein tuning the wavelength of the optical energy emitted by the nanoscintillators comprises doping of the nanoscintillators.

18. The method of claim 16, wherein tuning the wavelength of the optical energy emitted by the nanoscintillators comprises tuning the dimensions of the nanoscintillators.

19. The method of claim 16, wherein tuning the wavelength of the optical energy emitted by the nanoscintillators comprises tuning the electronic band structure of the nanoscintillators.

20. The method of claim 1, wherein introducing a plurality of nanoscintillators into a region with light sensitive cells comprises introducing a first plurality of nanoscintillators into a first sub-region with light sensitive cells, and introducing a second plurality of nanoscintillators into a second sub-region with light sensitive cells.
